(19) United States
(12) Patent Application Publication
(10) Pub. No.: US 2008/0125815 A1
(43) Pub. Date: May 29, 2008

(54) SYSTEM AND METHOD FOR ATTACHING SOFT TISSUE TO BONE

(75) Inventors: Malcolm Heaven, Dana Point, CA (US); Michael Ko, Mission Viejo, CA (US)

Correspondence Address: KNOBBE MARTENS OLSON & BEAR LLP, 2040 MAIN STREET, FOURTEENTH FLOOR, IRVINE, CA, 92614, US

(73) Assignee: KFx Medical Corporation, Carlsbad, CA (US)

(21) Appl. No.: 11/937,450

(22) Filed: Nov. 8, 2007

Related U.S. Application Data
(60) Provisional application No. 60/858,622, filed on Nov. 13, 2006.

(51) Int. Cl. A61B 17/56; A61B 17/04; A61B 17/58

(52) U.S. Cl. 606/308; 606/232; 606/104

(57) ABSTRACT

Disclosed herein are methods and devices for securing soft tissue to a rigid material such as bone. A non-metallic bone anchor is described that comprises a base and a top such that suture material may be compressed between surfaces on the base and top to secure the suture to the anchor by ultrasonically welding the surfaces. Also described is an inserter/welder that can be used to insert the bone anchor into bone and move the anchor top relative to the anchor base to clamp suture material there between and to weld the suture material within the clamping area.

Cross Reference to Related Applications

This application claims the benefit of U.S. Provisional Application No. 60/858,622, filed on Nov. 13, 2006, the disclosure of which is incorporated by reference herein in its entirety.

### BACKGROUND OF THE INVENTION

1. Field of the Invention

The present invention relates to medical devices and procedures. More particularly, the present invention relates to devices and methods for securing soft tissue to a rigid material such as bone.

2. Description of the Related Art

There are several medical procedures where a surgeon needs to attach soft tissue such as tendons or other soft connective tissue to bone. One common example is a torn rotator cuff, where the supraspinatus tendon has separated from the humerus causing pain and loss of ability to elevate and externally rotate the arm. To repair a torn rotator cuff, typically a surgical procedure is used to suture the torn tendon to the bone using a variety of methods. Some procedures utilize large incisions and involve complete detachment of the deltoid muscle from the acromion. Small diameter holes are made in the bone for passing suture material through the bone to secure the tendon. Such large incision procedures are traumatic, causing prolonged pain and recovery time. Other procedures make small incisions and use arthroscopic techniques to attach sutures using either small diameter holes or a bone anchor. However, it is difficult to manipulate sutures within the surgical site using arthroscopic techniques. In addition, when knot tying is used to secure the suture to a bone anchor, it is difficult to properly adjust the tension of the suture while tightening the knot. Similarly, when the suture is attached to a bone anchor prior to insertion of the anchor into the bone, it is difficult to judge the appropriate point of attachment so that the suture will be properly tensioned upon insertion of the bone anchor into the bone. Thus, there is a need for methods and devices that allow easy arthroscopic attachment of a suture to a bone anchor after the anchor is inserted into the bone without the use of knot tying.

### SUMMARY OF THE INVENTION

One embodiment provides for an anchor which may be used to secure a suture to bone. The anchor may include an anchor top having a welding feature, and may further include an anchor base which receives the anchor top. In some embodiments, the anchor top may include a portion that is non-metallic. The anchor base may also include a portion of the anchor base that is non-metallic. In some embodiments, the anchor top and anchor base are made out of polyether ether ketone (PEEK). The anchor top may also be part of a suture securing mechanism positioned proximally relative to the anchor base. When the anchor top is coupled to the anchor base, the suture securing mechanism may be adapted to receive and secure a suture moved laterally into the mechanism.

In another embodiment, a method of attaching soft tissue to bone is provided. The method includes passing a length of suture over the soft tissue and inserting an anchor into the bone. Once the anchor has been inserted into the bone, the length of suture may be secured to the anchor by ultrasonically welding the suture to the anchor.

### BRIEF DESCRIPTION OF THE DRAWINGS

FIG. 1 depicts attaching soft tissue to bone using a single bone anchor and a stitch.

FIG. 2 depicts attaching soft tissue to bone using a two bone anchors with a suture stretched there between.

FIGS. 3A-3C depict various geometries of bone anchors and suture patterns for attaching soft tissue to bone.

FIGS. 4A-4C depict the base of a two-part non-metallic suture anchor that can be inserted into bone.

FIGS. 5A-5D depict the top of a two-part non-metallic suture anchor.

FIGS. 6A, 6B and 6C depict the suture anchor top of FIGS. 5A-5C inserted into the suture anchor bottom of FIGS. 4A-4D.

FIGS. 7A and 7B depict a handheld ultrasonic welding device.

FIGS. 8A-8D depict a suture anchor inserter and a combination inserter/welding device.

FIGS. 9A-9F depict manipulation of a non-metallic suture anchor using a suture anchor inserter to insert the suture anchor into bone and attach suture material to the suture anchor by welding the anchor top to the anchor base.

FIG. 10 depicts the top of an alternate non-metallic two-part suture anchor with welding features in the proximal member and the tip.

FIGS. 11A-11D depict a non-metallic anchor top with an anvil and a non-metallic anchor base with crimp sleeves.

FIGS. 12A-12D depict a non-metallic anchor top with a floating cap.

FIGS. 13A-13B depict an alternate embodiment of an anchor system.

### DETAILED DESCRIPTION OF THE CERTAIN EMBODIMENTS

In various embodiments, soft tissue may be attached to bone utilizing one or more non-metallic bone anchors with suture attached thereto. As used herein, “suture” refers to any flexible structure that can be stretched between two or more anchors and includes, without limitation, traditional suture material, single or multiple stranded threads, or a mesh structure. A suture may also be a strap-like structure with a number of holes in it, similar to the holes found in a belt. A “suture” may also take the form of an acellular, collagen membrane or other biologic tissue augment such as described in U.S. Application Publication No. 2006/0067967, which is incorporated herein by reference in its entirety, which may provide a scaffold or support matrix for cellular ingrowth to allow soft tissue to reconstruct itself. Suitable biologic tissue augments that are commercially available include, but are not limited to, those available under the trade names TISSUEMEND® (TEI Biosciences Inc., Boston, Mass.), RESTORE® (Depuy, Warsaw, Ind.), GRAFT JACKET(® (Wright Medical, Arlington, Tenn.), and CUFF PATCH™ (Organogenesis Inc., Canton, Mass.). The membrane may be used in conjunction with other types of sutures to provide additional support in areas where the tissue is weakened. The augment may also be used to bridge gaps or span a defect between soft tissue including ligaments and tendons as well as gaps between the ligament or tendon to bone insertion points.

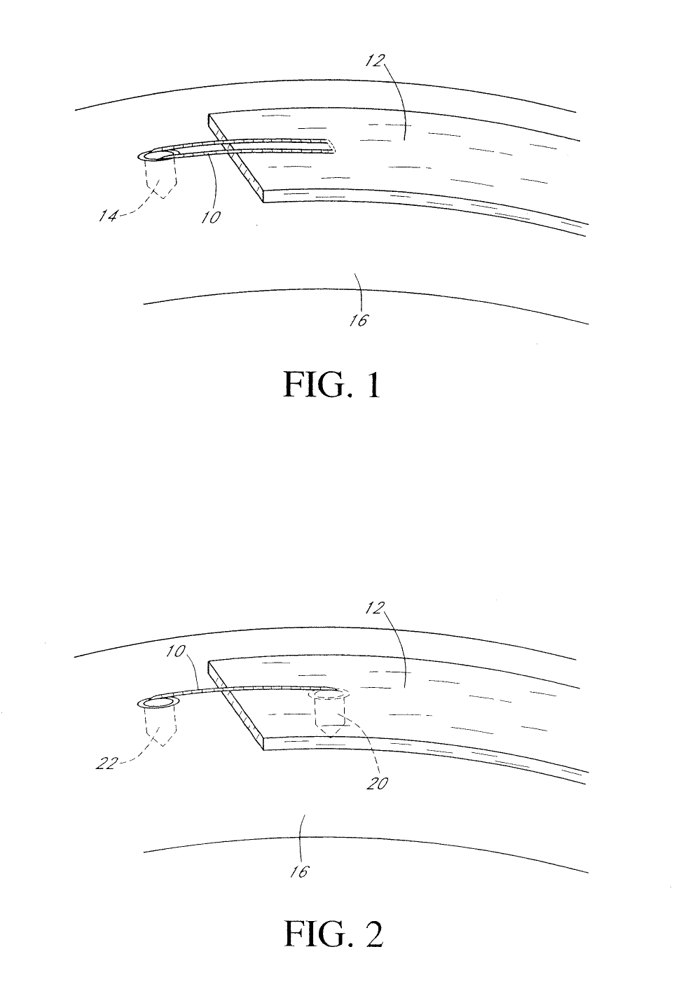

In some embodiments, suture is passed over the top of the soft tissue so that the suture can press the soft tissue against the bone. In one embodiment, a length of suture is attached to a single bone anchor. One non-limiting example, depicted in FIG. 1, includes stitching the suture 10 to the soft tissue 12, such as by an incline mattress stitch, and then securing the suture 10 to the single bone anchor 14 that is inserted into the bone 16. However, in other embodiments, a length of suture is attached to multiple bone anchors. The use of multiple bone anchors increases the footprint over which the suture material presses the soft tissue against bone. One non-limiting example, depicted in FIG. 2, includes two bone anchors. One anchor 20 is positioned in a medial location underneath the soft tissue 12 and a second anchor 22 is positioned lateral to the soft tissue 12. The suture 10 is attached to both anchors.

In one embodiment, the suture 10 is attached to the lateral bone anchor 22 only after the medial bone anchor 20 is inserted and the suture 10 is passed over the soft tissue 12. In one embodiment, the suture 10 is attached to the medial bone anchor 20 prior to insertion of the medial bone anchor 20. Thus, in this embodiment, the surgeon does not need to pass the suture through the soft tissue 12 from beneath the soft tissue 12. In one embodiment, the procedure involves inserting the medial bone anchor 20 with suture 10 pre- attached through the soft tissue 12. The medial bone anchor 20 may then be moved laterally relative to the bone 16 in order to pull the soft tissue 12 laterally relative to the bone 16. After appropriate positioning of the soft tissue 12, the medial bone anchor 20 may then be inserted into the bone 16. The lateral bone anchor 22 may then be inserted into the bone 16. The suture 12 may then be passed over the soft tissue 12 and attached to the lateral bone anchor 22. In some embodiments, a lateral bone anchor 22 is provided to which suture 12 can be attached without tying any knots or without passing the suture 12 through any aperture in the lateral bone anchor 22.

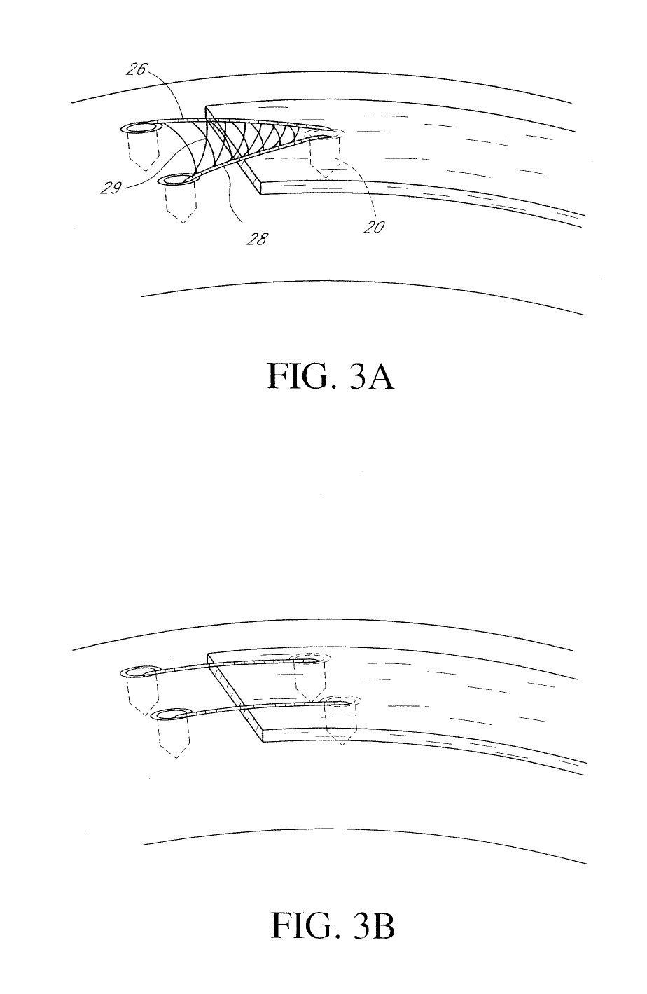

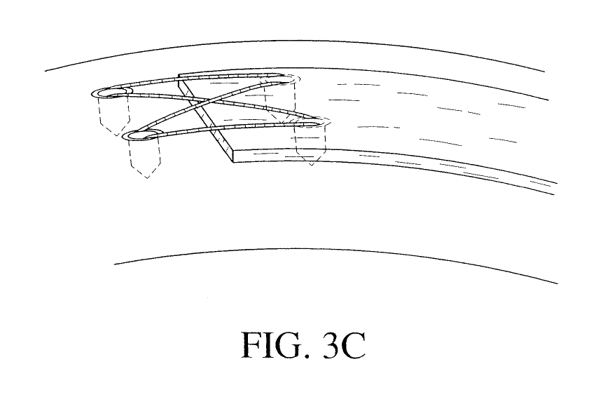

In some embodiments, multiple anchors and multiple suture lengths may used to provide a wider area of pressure of the soft tissue against bone. For example, as depicted in FIG. 3A, three anchors are used with two lengths of suture 26 and 28. Alternatively, a mesh structure 29 may be stretched between the three anchors. In another example, as depicted in FIG. 3B, four anchors are used with two lengths of suture. In still another example, as depicted in FIG. 3C, four anchors are used with four lengths of suture. In some embodiments, the individual suture lengths may be part of a larger continuous suture. For example, in FIG. 3A, the suture lengths 26 and 28 may be part of a larger length of suture such that the lengths 26 and 28 are joined at medial bone anchor 20. Those of skill in the art will appreciate that there are any number of anchor and suture geometries that can be used.

In some embodiments, the medial bone anchors 20 are designed so that they can be easily pierced through the soft tissue 12 and bone 16. In some embodiments, the lateral bone anchors 22 are designed so that they can easily capture suture material after insertion of the bone anchors 22. Together, these design features provide a suturing system and method that provides an increased footprint of suture pressure against the soft tissue 12 and ease of implementation for a surgeon. For example, in some embodiments, the entire procedure may be done arthroscopically, with the surgeon needing only to insert the medial bone anchor 20 with suture optionally pre-attached through a first port, insert the lateral anchor 22 through a second port, pass the suture over the soft tissue 12 by capturing it from within the second port, and securing the suture to the lateral anchor 22. Accordingly, described below are certain embodiments of anchors adapted to capture suture material and anchors adapted to easily pierce through soft tissue and bone. Further description of suitable anchors and methods are provided in U.S. application Ser. No. 11/143,007, filed on Jun. 1, 2005 and published as Publication No. 2006-0004364, and U.S. Provisional Application No. 60/753,445, filed on Dec. 22, 2005, both of which are incorporated herein by reference in their entirety.

### Non-Metallic Suture Capturing Anchor

One embodiment is a non-metallic bone anchor that allows easy capturing and securing of a suture after the bone anchor is inserted into the bone. The anchor may be constructed wholly or in-part of a non-metallic substance that is biocompatible. In one embodiment, the substance may be a biocompatible engineering plastic such as polyether-ether-ketone (PEEK). Alternatively, biocompatible materials such as poly ether ketone (PEK), polyethermide (ULTEM), ultrahigh molecular weight polyethylene (UHMPE), or some other engineering polymer materials known to those of skill in the art may be used. A non-metallic anchor system may provide certain advantages such as, for example, eliminating MRI artifacts. In addition, due to the pliant nature of many biocompatible engineering plastics, the parts of the anchor system may be more effectively secured together by welding the plastic parts using a device such as an ultrasonic welding device.

In one embodiment, the bone anchor includes a suture securing mechanism positioned on the proximal end of the bone anchor (i.e., the end nearest the surface of the bone and the surgeon). In one embodiment, the suture securing mechanism allows a suture to be moved laterally into the mechanism, By “laterally,” it is meant that the suture can be moved into the mechanism by moving the suture in a direction that is generally perpendicular to the axis of the suture. In other words, the suture can be moved into the mechanism without threading an end of the suture into the mechanism. In one embodiment, the suture can be fixedly secured within the mechanism without tying any knots. By “fixedly secured,” it is meant that the suture within the securing mechanism cannot be easily moved relative to the bone anchor.

In one embodiment, the non-metallic bone anchor consists of two parts: an anchor base and an anchor top. The anchor base may be designed to be inserted into a hole in the bone with a proximal surface facing up. The anchor top may be coupled to the anchor base via a distal member. A proximal member on the anchor top may have a distal surface facing down toward the proximal surface on the anchor base. The coupling of the anchor top to the anchor base may be such that the anchor top can move relative to the anchor base such that it can be positioned in one configuration where there is space between the proximal surface on the anchor base and the distal surface on the proximal member of the anchor top. In another configuration, the proximal member of the anchor top may be positioned such that there is very little space, if any, between the proximal surface on the anchor base and the distal surface on the proximal member of the anchor top. Thus, in the first configuration, suture material may be easily passed between the two surfaces and tensioned as desired. In the second configuration, the suture material may be clamped between the two surfaces such that the suture is secured to the bone anchor.

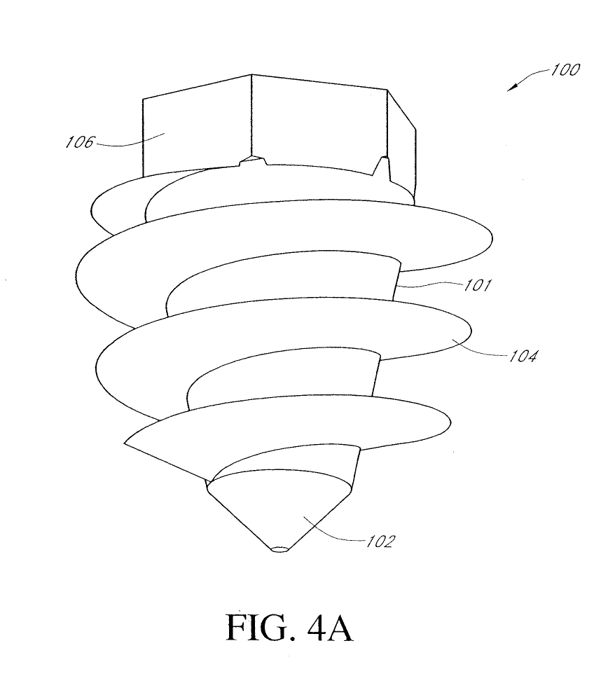

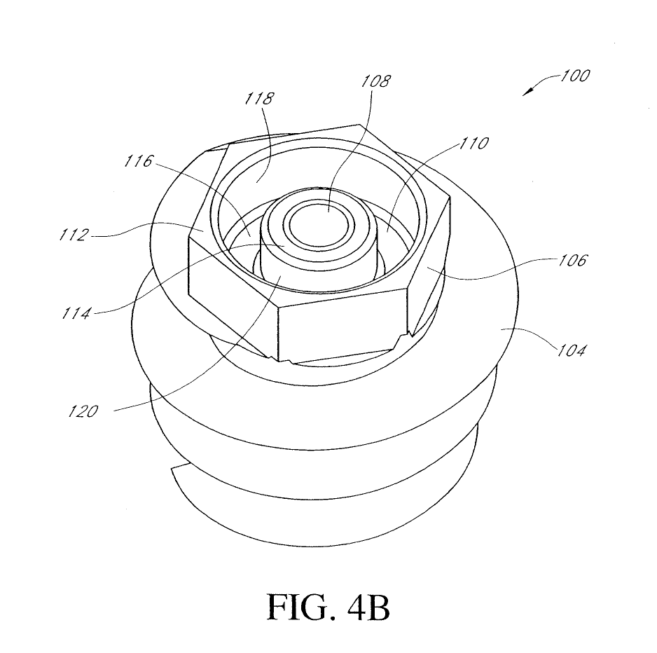

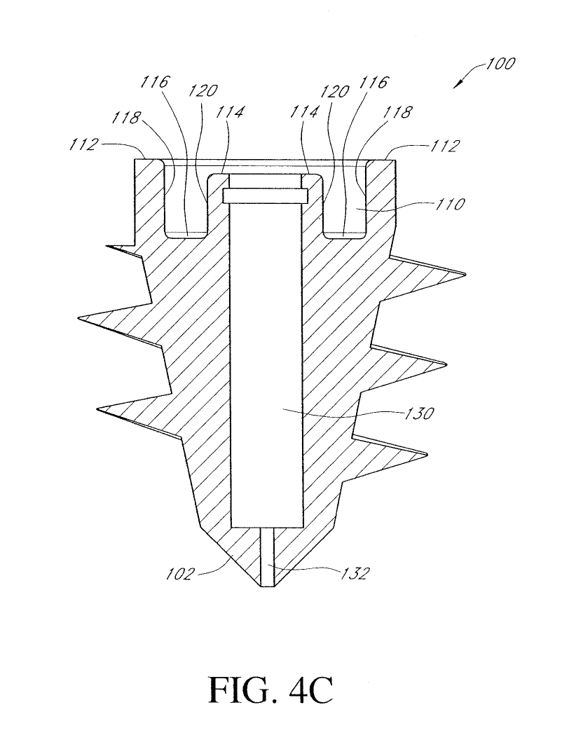

One embodiment of a non-metallic anchor base 100 is depicted in FIGS. 4A through 4D. FIG. 4A is a perspective view showing the side 101 and bottom 102 of the anchor base 100. The bottom 102 of the anchor base 100 may advantageously be tapered to facilitate insertion of the anchor base 100 into bone. In some embodiments, a hole is predrilled into the bone to facilitate insertion of the anchor base 100. In other embodiments, the anchor base 100 is forced directly into the bone, thereby creating the hole. The sides 101 of the anchor base 100 comprise threads 104 so that the anchor base 100 may be inserted into bone using a screwing action. In some embodiments, the anchor base 100 may be tapped to start the threads 104 into the bone followed by screwing the anchor base 100 into the bone. When the hole in the bone is pre-drilled, the hole is advantageously drilled with a diameter smaller than the diameter of threads 104 so that the threads engage the bone through the sides of the hole. It will be appreciated that means other than threads may be used to secure the anchor base 100 to bone. For example, angled protrusions may be used that provide greater resistance to removal of the anchor base 100 than to insertion. The protrusions may be static or deployable once the anchor is inserted.

The top of anchor base 100 may include a structure 106 for facilitating the driving or screwing of the base 100 into the bone. In the illustrated embodiment, this comprises a hexagon nut structure 106 that facilitates engagement with a hex nut driver for screwing the anchor base 100 into the bone. It will be appreciated that other structures known in the art for engaging tools used for screwing action may be used instead of hex nut structure 106, and that this structure can be indented into or extending out from the top of the anchor base 100, or can alternatively be formed on the sides of the anchor base 100.

With reference to FIG. 4B, which is a perspective view of the top and side of the non-metallic anchor base 100, the top (proximal end) comprises a hole 108 in the center for receiving the anchor top, which is described below. The top of anchor base 100 may also contain a suture gripping structure such as a circular groove 110 that may be concentric with hole 108. Because of groove 110, the proximal surface of anchor base 100 is not flat and comprises top surfaces 112 and 114, bottom surface 116, and side surfaces 118 and 120. In some embodiments, the suture gripping structure may be relatively smooth, relying on a welding process (described in further detail below) to provide gripping of suture material. In other embodiments, some or all of these surfaces may be textured or otherwise altered so as to inhibit movement of suture material pressed against the surfaces. Suitable structures for inhibiting suture movement include, but are not limited to, teeth on the surfaces, bead blasting of the surfaces, etching the surfaces such as through a chemical etch process, use of a rubber o-ring such as within the groove 110, and various coatings such as a hydroxyapatite coating, grit coatings, or microencapsulated biocompatible adhesives. Although a grooved surface is illustrated, it will be appreciated that other shapes for the proximal surface of anchor base 100 are also contemplated, including multiple concentric grooves, a series of protruding ridges or welding features, a “vee” shaped channel, or any other suitable structure that permits a suture to be securely locked against the top or proximal end of the anchor base 100.

Hole 108 in anchor base 100 is an opening into a central (“axial”) bore into the anchor base 100. FIG. 4C depicts a cross section through the center of the non-metallic anchor base 100. This view illustrates central bore 130 and groove 110. The proximal surfaces 112, 114, 116, 118, and 120 are also apparent. Central bore 130 may not extend all the way through the anchor base 100. Instead, a smaller bore 132 may be present at the distal end 102 of the anchor base 100. Smaller bore 132 may be used to receive a wire connected to an anchor inserter. It will be appreciated that other structures than the smaller bore 132 may be used for attaching wire. For example, the anchor base 100 may include a blind hole with a cutout for attaching the wire. In addition, other means than a wire may be used to secure the anchor to the anchor inserter as will be discussed in further detail below.

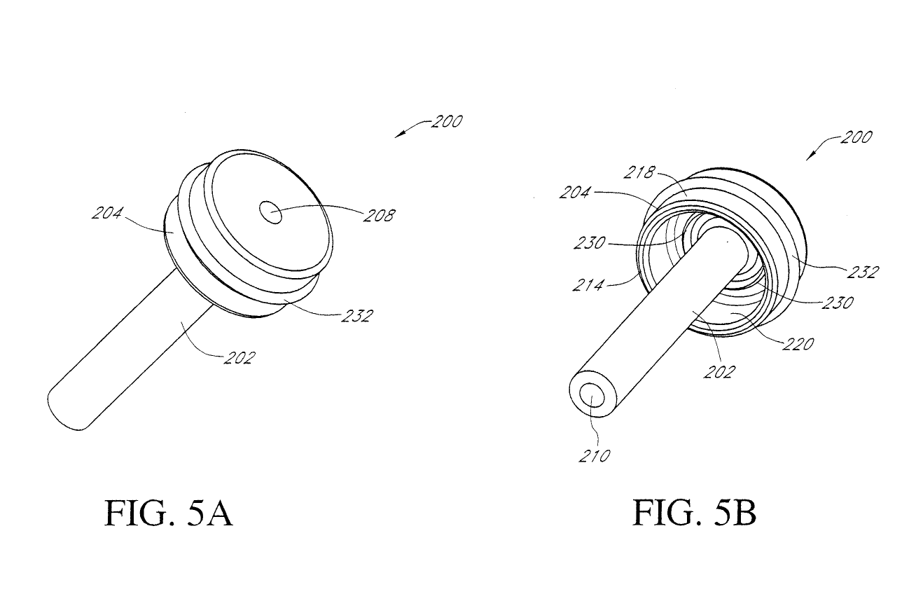

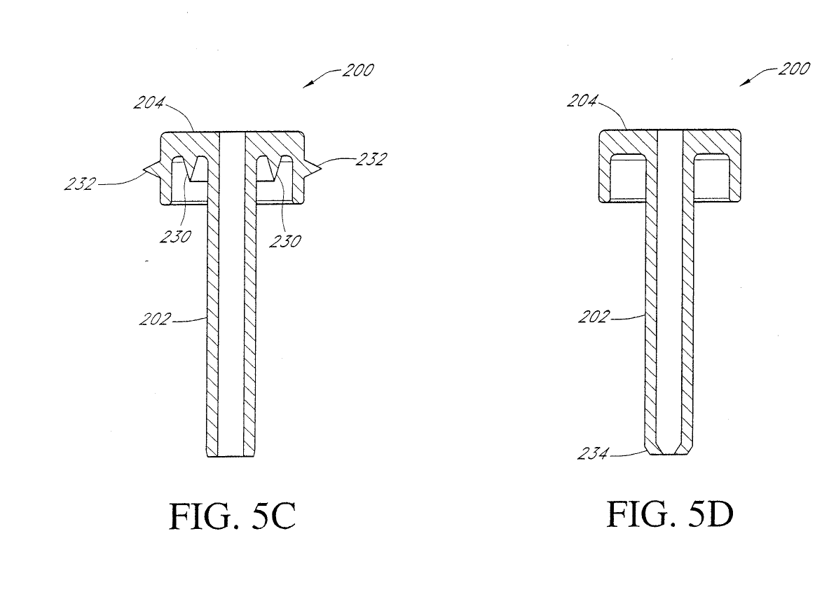

FIGS. 5A through 5C illustrate one embodiment of a non-metallic anchor top 200. FIG. 5A provides a perspective view of the side and top of the anchor top 200 and FIG. 5B provides a perspective view of the side and bottom of the anchor top 200. Anchor top 200 has two members, a distal member 202 and a proximal member 204. The distal member 202 comprises an elongated shaft, the longitudinal direction of which shall be considered to run along the axis of the distal member 202. The distal member 202 is designed to be inserted into the central bore 130 of the non-metallic anchor base 100.

The proximal member 204 of anchor top 200 is generally cylindrical in shape with a diameter larger than distal member 202. A hole 208 may advantageously be provided in the center of proximal member 204. With reference to FIG. 5B, the bottom of distal member 202 may also contain a hole 210. Holes 208 and 210 open into a central bore through the anchor top 200. This central bore allows the wire referred to above to extend through the anchor top 200 to be secured to the bore 132 in the anchor bottom 100, thus allowing the anchor bottom 100 to be attached to an anchor inserter while still allowing anchor top 200 to be inserted into anchor bottom 100. In some embodiments, a wire is not used to attach the anchor to an anchor inserter, in such embodiments, the anchor top 200 may lack holes 208 and 210 and the central bore.

FIG. 5B also illustrates that proximal member 204 may contain one or more interior welding features 230 which protrude from the interior distal surface of the proximal member 204 of the non-metallic anchor top 200. For example, the welding features 230 depicted in FIG. 5B consist of a ring that tapers to a point. However, any suitably shaped protrusion may be used. The non-metallic anchor top 200 may also include one or more exterior welding features 232 which protrude from the exterior distal surface of proximal member 204. Suitable attachment of the anchor top 200 to the anchor base 100 may be achieved by welding the welding features 230, the external welding features 234, or both. The protruding configuration of the interior welding features 230 and the exterior features 232 which may help in capturing a suture provides a greater surface area and volume of non-metallic material to bind with the anchor base 100 when it flows due to ultrasonic vibration from an ultrasonic welding unit. Thus, the distal surface of proximal member 204 is not flat and comprises interior welding features 230 and exterior welding features 232 and side facing surfaces 218 and 220. FIG. 5C depicts a cross section through the center of anchor top 200. In this figure, the central bore 226 is depicted as are welding features 230 and 232.

FIG. 5D depicts a cross section of an alternative non-metallic anchor top 200 which can be secured to a non-metallic anchor base 100 through the use of welding features located in the distal area of distal member 202. In this embodiment, tip welding features 234 are located in the distal area of the distal member 202, which may be welded to the interior of the central bore 130 of the anchor base 100. Because the tip can be welded to any portion of the central bore 130, the distal member 202 in the non-metallic anchor top 200 may be shortened and still provide the necessary adhesion to hold the anchor top 200 securely within the central bore 130. Shortening the length of the shaft may be advantageous in that it allows for better maneuverability in constricted spaces, and it also may provide a further advantage in reducing material costs because the shorter shaft will need less material to construct.

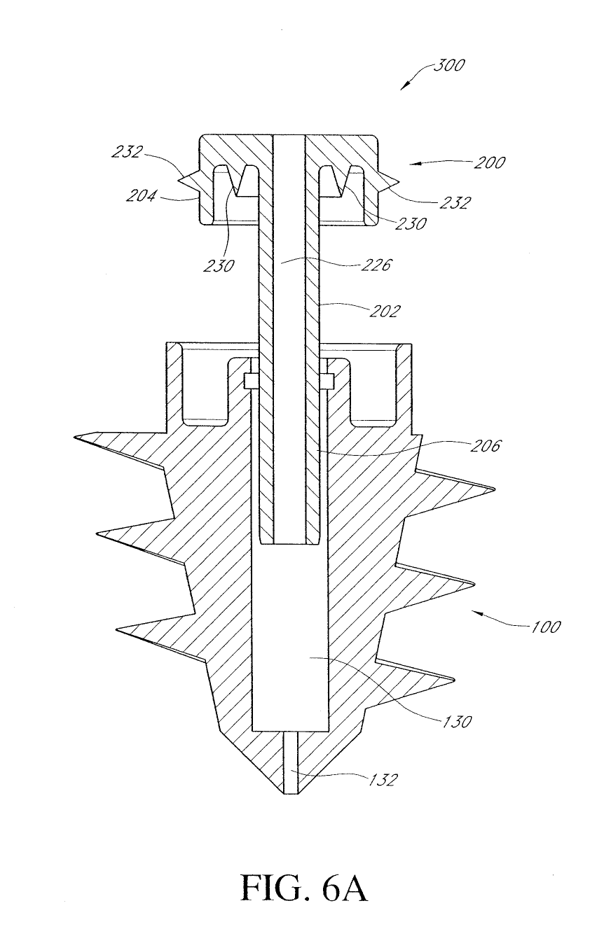

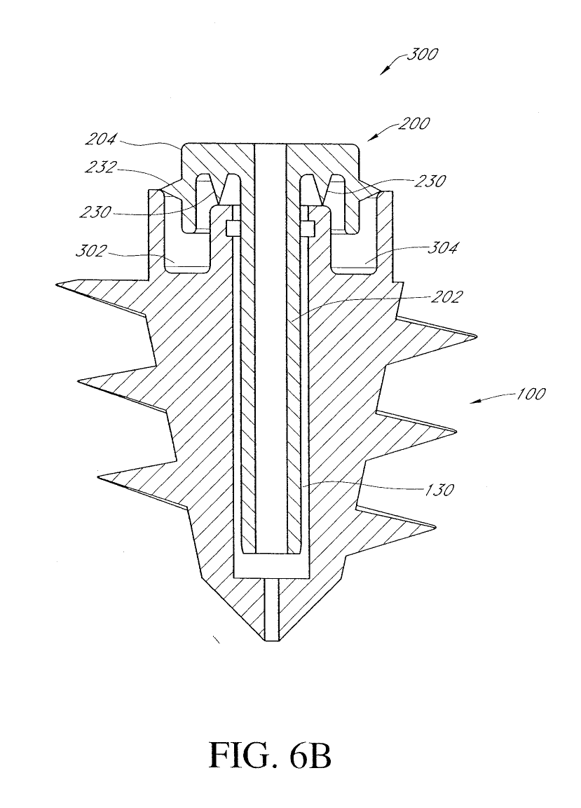

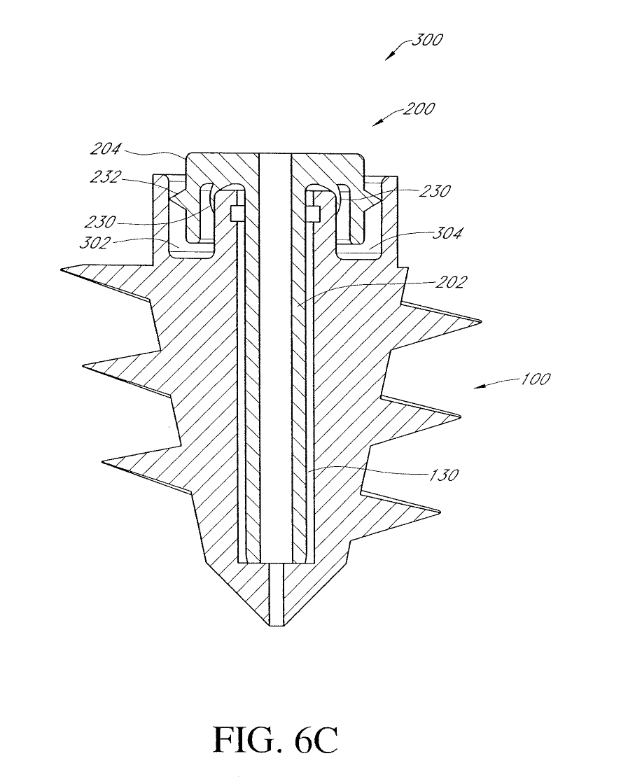

FIGS. 6A-6C depict cross sections showing how the anchor top 200 may be coupled to anchor base 100 to form the complete anchor 300. In FIG. 6A, the anchor top 200 is coupled to anchor base 100 with the proximal member 204 separated from the anchor base 100. This separation allows for suture material to be positioned between the proximal member 204 of the anchor top and the top of the anchor base. In order to maintain an appropriate separation for positioning the suture material, the distal member 204 of the anchor top 200 may be sized with so that it forms a snug fit into the central bore 130 of the anchor base 100 that requires a degree of force to push it further into the central bore. In one embodiment, the distal member may include a circumference that is 1/1000 of an inch larger than the circumference of the central bore 130. Alternatively, the distal member 204 may have a circumference slightly smaller than the central bore, but may include molded features on the exterior surface that extend beyond the circumference of the central bore 130 to provide friction that maintains the position of the anchor top 200 relative to the anchor base 100. When downward pressure is applied to the molded features, they may collapse into the central bore. In another embodiment, the interior welding features 130 could extend out of the distal surface of the proximal member of the anchor top 200 to maintain appropriate separation by resting against the proximal surface of the anchor base 100. It should be appreciated that any suitable structure for holding the anchor top away from the anchor base may be used.

FIG. 6B depicts the anchor assembly 300 with the welding features 230 of the anchor top 200 positioned against the proximal surface of the anchor base I 00. Because the welding features 230 extend outward from the distal surface of the proximal member 204, the anchor top cannot be inserted fully into the central bore 130 of the anchor base 130 because the welding features abut against the proximal surface of the anchor base 100.

FIG. 6C depicts the anchor assembly 300 with the distal member 202 of anchor top 200 fully inserted into central bore 130 in anchor base 100 after the proximal surface of the anchor top 200 has been touched by a ultrasonic welding device. The welding device causes the welding features to flow (as will be discussed in further detail below), which allows the anchor top 200 to be fully inserted into the anchor base. In this fully inserted configuration, it can be seen that proximal surfaces 112, 114, 116, 118, and 120 of the anchor base 100 and distal surfaces 214, 216, 218, and 220 of the proximal member 204 of anchor top 200 form passageways 302 and 304. The size of passageways 302 and 304 may be advantageously configured such that when a suture passes through them, it will be compressed so that it is securely attached to the anchor 300. The suture may also be further secured by the flowing plastic infiltrating the interstices of the suture and re-solidifying therein. In one alternative embodiment, the suture may include a strap-like or belt-like structure with holes similar to those found in a belt. The distal member 202 of the anchor top 200 suture may be passed through one of the holes to provide appropriate tensioning of the suture, and may then be fully inserted welded to the anchor base 100 to secure the suture to the anchor 300.

Various techniques may be utilized to seat the anchor top 200 within the anchor base 200. In one embodiment, the anchor top 200 may be seated within the anchor base through the application of pressure from the tip of an ultrasonic welding device to the proximal surface of the anchor top 200 to push it down into the anchor base where it may then be welded to further secure the base to the anchor top.

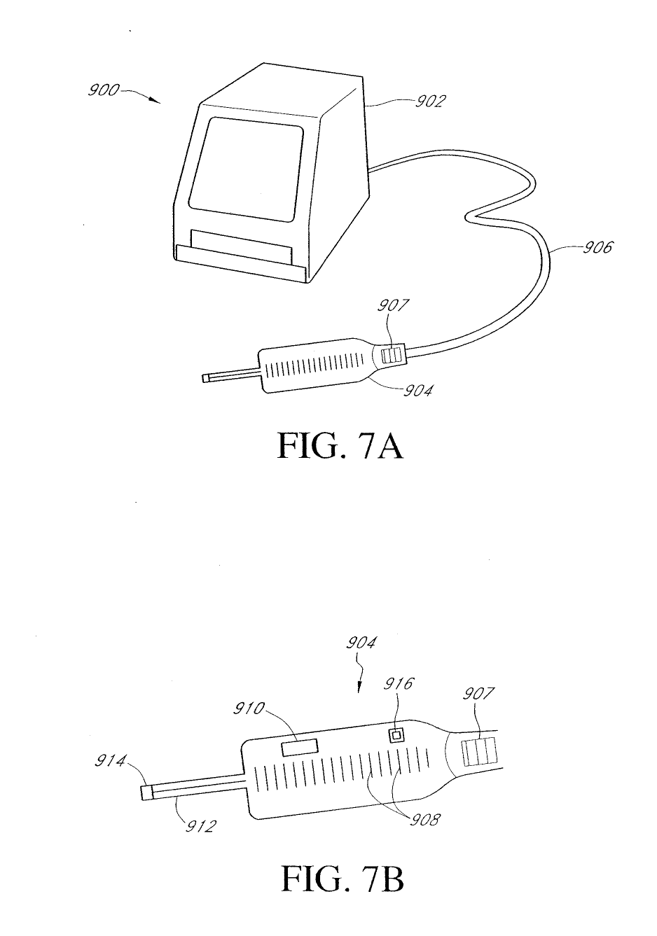

Referring now to FIG. 7A, an example of an ultrasonic welding device 900 is provided that may be used to apply pressure to the anchor top and weld the anchor top to the anchor base is provided. The welding device 900 may be used to weld the welding features of the non-metallic anchor top 200 to appropriate areas of non-metallic anchor base 100. Although non-metallic anchor top 200 and non-metallic anchor base 100 may be constructed of the same material, one of skill in the art will appreciate that an ultrasonic welding device may be used to weld both similar and dissimilar non-metallic substances together. Thus, the anchor top 200 may be constructed of a first non-metallic substance, while the anchor base 100 may be constructed of a second non-metallic substance. In other embodiments, both anchor base 100 and anchor top 200 may include more than one type of thermoplastic material, so long as the welding features of non-metallic anchor top 200 can be suitably welded to receiving areas in the anchor base 100. In other embodiments, the anchor base 100 and anchor top 200 may include both metallic and non-metallic parts. For example, with reference to FIG. 5C, only welding feature 232 and a corresponding feature on the anchor base 100 may be non-metallic.

The ultrasonic welding device 900 may be a portable unit providing sufficient mobility to be used to spot weld materials within the limited space generally available during surgical procedures. Ultrasonic welding device 900 may include a power supply 902 to generate sufficient ultrasonic vibrations to melt the plastic materials. The power supply 902 may be electronically powered by plugging it into a typical wall outlet, or in some instances it may be powered by a battery. The ultrasonic welding device 900 may further include a lightweight handgun 904 which may be pressed against the welding features and receiving parts. While pressed against the welding features and receiving parts, the handgun transfers the ultrasonic energy, which causes the welding features to flow and bond or meld. The handgun 904 may be tethered to the power supply 902 by a cord, tube or wire 906 which carries the energy from the power supply 902 to the handgun 904. In one embodiment, the power supply 902 includes a transducer which converts the electricity into ultrasonic energy which is carried by a sound-conductive tube 906 to the handgun 904. Alternatively, the handgun 904 may include a transducer 907 (as shown in FIG. 7A), and the cord 906 may transmit electrical energy to the handgun 904 which is then converted to ultrasonic energy by the transducer 907.

FIG. 7B depicts a more detailed view of the handgun 904. The handgun 904 may include gripping ridges 908 which allow the operator of the handgun to more securely grip the device. As discussed above, the handgun 904 may also include a transducer 907 that converts electrical energy to ultrasonic energy. The handgun 904 may also include a lid 910 to allow access to the internal components of the handgun 904 for service or repair. The handgun 904 may also include an elongated horn 912 which includes a tip 914 which may be pressed against the target material to transfer the ultrasonic energy for welding. The handgun 904 may also include a trigger or toggle switch 916 which may be used to activate and deactivate the ultrasonic pulses when the tip is pressed against target material.

In an alternative embodiment, the anchor top 200 may be seated within the anchor base 100 by utilizing a draw wire which extends into the top of the anchor 300 and is secured to bore 132 (see FIG. 4C). The wire may then be coupled to an anchor inserter 400, and it can hold the entire anchor assembly 300 and still allow anchor top 200 to move relative to anchor base 100 and the wire.

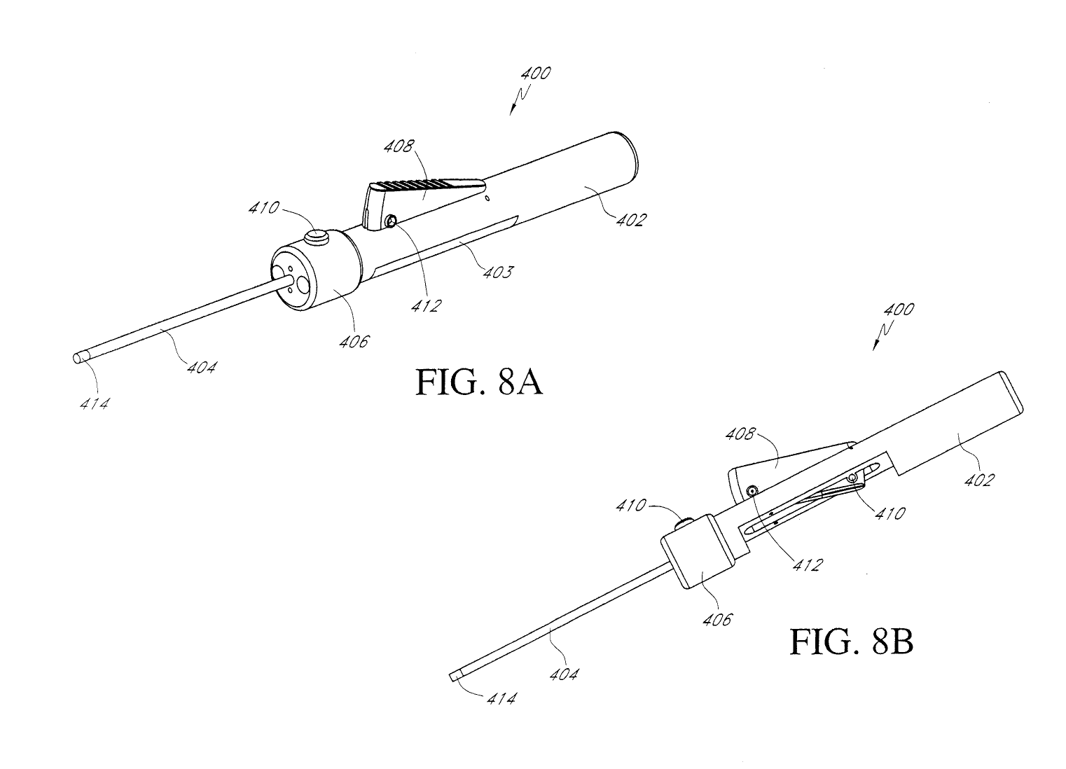

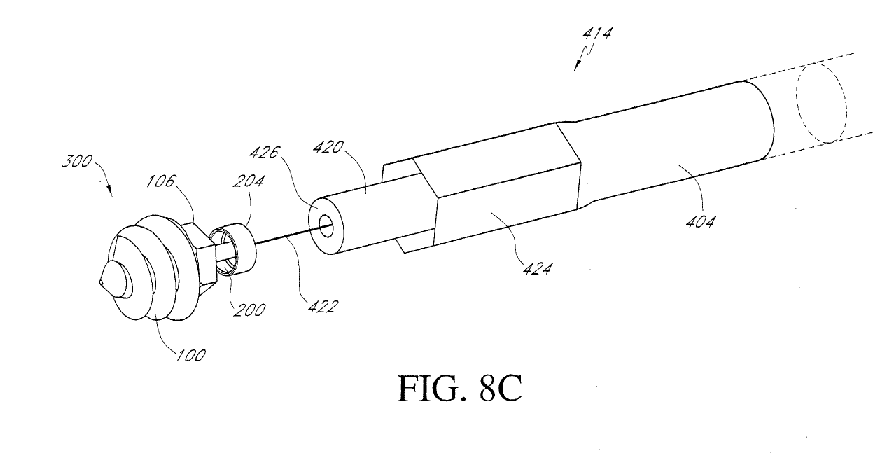

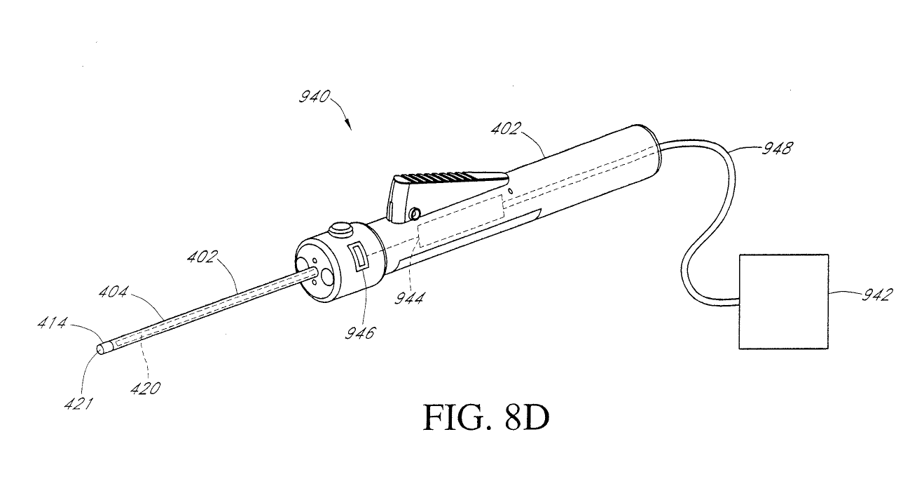

One such anchor inserter 400 is depicted in FIGS. 8A and 8B. Inserter 400 comprises a handle 402 and an outer tube 404. As depicted in FIG. 8A, the handle 402 comprises a cover 403. FIG. 8B depicts the inserter 400 with cover 403 removed. Not depicted in FIGS. 8A and 8B are an inner tube disposed inside outer tube 404 and a wire disposed within the inner tube. As will be described in more detail below, the inner and outer tubes may be used to manipulate an anchor 300 such as that described in FIGS. 4-6. The wire may be used to couple the inserter 400 to the anchor 300 as described above. Inserter 400 also comprises an outer tube manipulator 406 and a wire manipulator 408. Outer tube manipulator 406 comprises release button 410. Outer tube manipulator 406 is securely attached to outer tube 404. Outer tube manipulator 406 may move longitudinally relative to handle 402 and the inner tube when release button 410 is pressed. Thus, when outer tube manipulator 406 is moved, outer tube 404 also moves.

Wire manipulator 408 comprises wire grabber 410 to which the wire is attached. The wire extends from wire grabber 410, through handle 402, and then through the inner tube. In one embodiment, wire manipulator 408 also comprises a release button 412. When release button 412 is pressed, the wire manipulator 408 may be pressed into the handle 402 to contact and thus provide additional tension on the wire. When in use, the additional tension causes the anchor base 100 to mover relative to inserter 400. When enough tension is provided to the wire by wire manipulator 408, the wire may break free from the anchor 300 at its attachment point in bore 132 or at some other predetermined location along the wire. It will be appreciated that any suitable breakable attachment means may be used for securing the wire to the anchor 300. For example, the wire may be frictionally secured into bore 132 or it may welded to the anchor base 100 using a weld that is weaker than the wire itself or a portion of the wire where breaking is desired may be weakened. In one embodiment, the wire is notched so as to create a weaker region in the wire that will break upon application of suitable force.

The tip 414 of outer tube 404 is depicted in more detail along with inner tube 420, wire 422, and anchor 300 in FIG. 8C. The end of outer tube 404 may comprise a hex nut driver structure 424 for receiving the hex nut structure 106 of anchor base 100. Of course, any other suitable engagement structure can be provided on the inserter 400 and the anchor base 100 in order to facilitate placement of the anchor base 100. Wire 422 extends out of inner tube 420 and into the central bore in the anchor top 200 to attach to anchor base 100 as described above. In some advantageous embodiments, the wire length and tension is adjusted such that the proximal member 204 of anchor top 200 buts against the end 426 of inner tube 420.

In yet another embodiment, features of the ultrasonic welding device 900 may be combined with features of the inserter 400 to provide a combined inserter/welder apparatus. This multipurpose tool may be used to insert and manipulate the anchor 300 and secure the non-metallic anchor cap 200 to the non-metallic anchor base 100 through ultrasonic welding.

FIG. 8D depicts an illustrative inserter/welder device 940. The inserter/welder device 940 may include a handle 402 and an outer tube 404. Within the outer tube, an inner tube 420 houses a wire used to couple the inserter/welder 940 to the anchor as described above in connection with FIGS. 8A-8C. Outer tube 404 also may include a tip 414 configured similarly to that described in FIG. 8C above with a hex nut driver structure (not shown) and may further include an ultrasonic tip 421, which may form part of the inner tube 420 that can be pressed against the proximal member 204 of the anchor top 200.

The inserter/welder device 940 may also include a power generator 942. The power generator 942 may generate ultrasonic energy and transmit the generated energy through a sound conductive tube 948 to the ultrasonic tip on the inner tube 420. Alternatively, the generator 942 may transmit electrical energy through wire 948 to an ultrasound transducer 944 located in the inserter/welder device 940. The ultrasonic energy may then be transmitted via sound conductive material in either outer tube 404 or inner tube 420 to the ultrasonic tip 421, which may then be applied to a target surface for ultrasonic welding. The inserter/welder device 940 may further include one or more triggers or toggle switches 946 to control the various functions that it performs. For example, the inserter/welder device 940 may include a power switch 946 for controlling the ultrasonic welding functions.

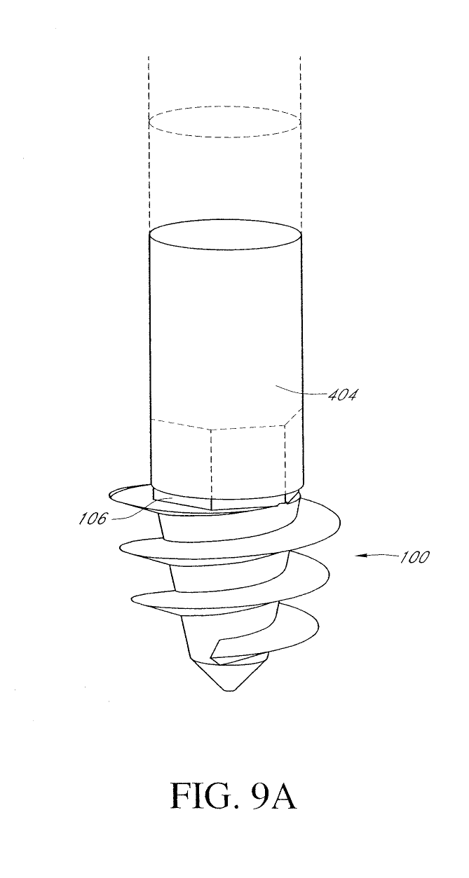

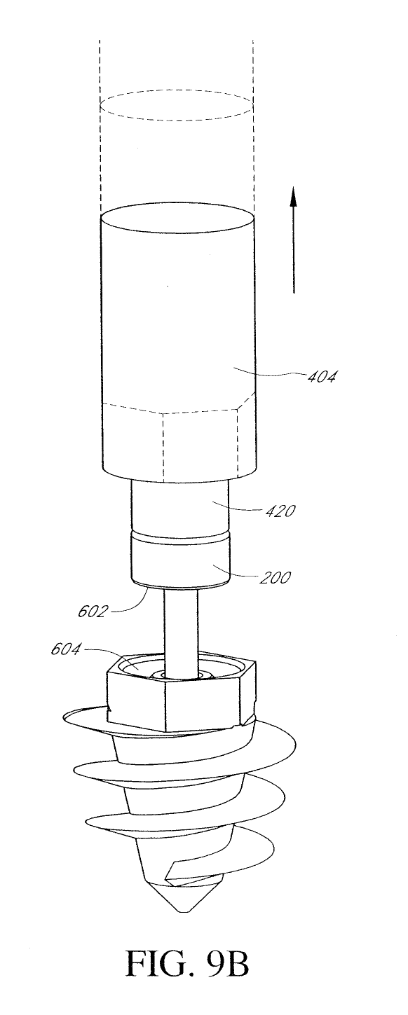

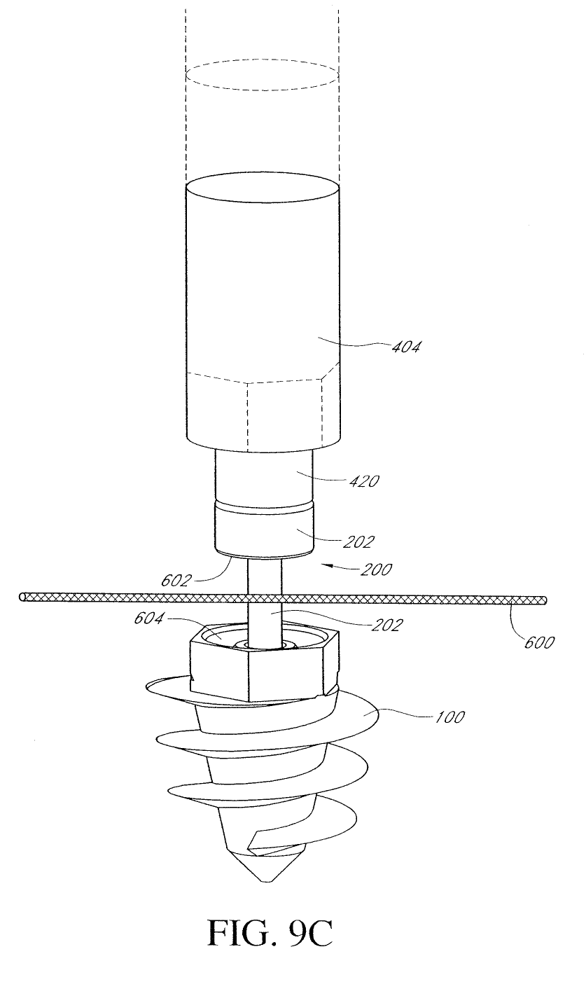

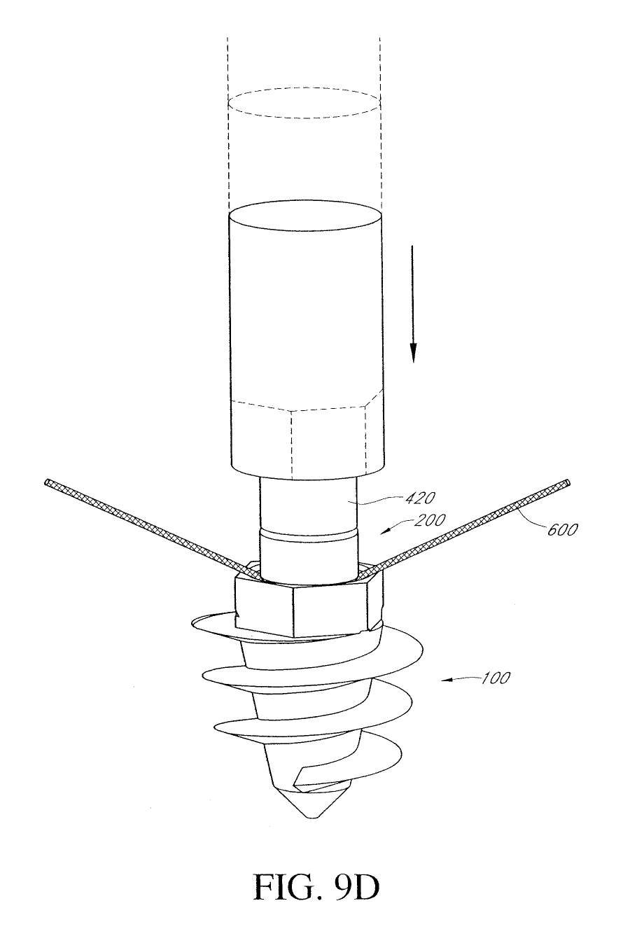

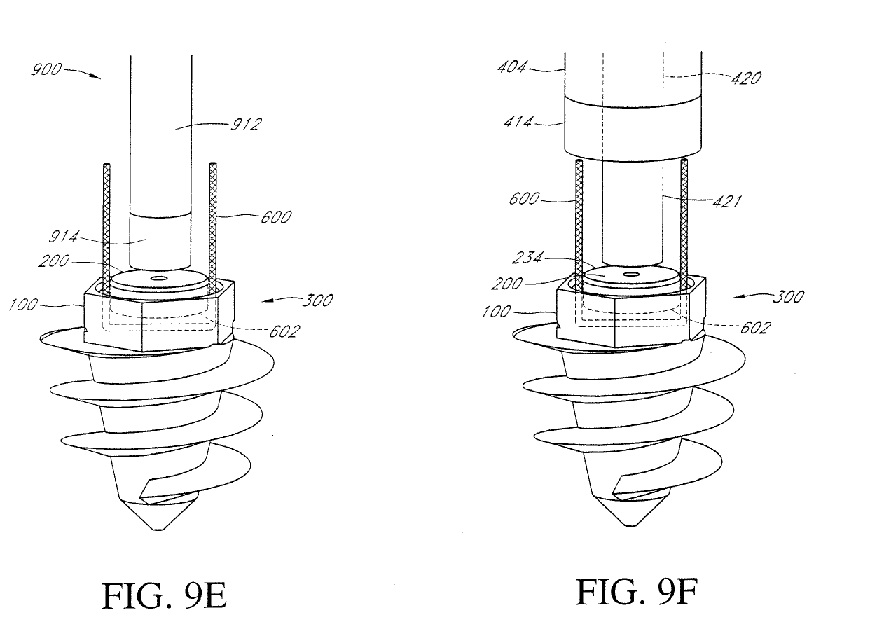

FIGS. 9A through 9F depict how an inserter 400, or alternatively, an inserter/welder device 940, may be used to insert the anchor 300 into bone and attach a suture to it. FIG. 9A depicts the configuration for inserting the anchor 300 (or more specifically, the anchor base 100) into bone. Outer tube 404 and outer tube manipulator 406 (see FIGS. 8A and 8B) are positioned relative to inner tube 420 and handle 402 (see FIG. 8C) so that the outer tube 404 engages hex nut structure 106 in the anchor base 100. The anchor top 200 may placed be in a position relative to the anchor base 100 using a wire as shown in FIG. 8C. Alternatively, the anchor top 200 may be not positioned relative to the anchor base 200, and instead be moved into position after the anchor base 100 has been inserted into the bone. In the configuration of FIG. 9A, a surgeon may screw the anchor base 100 into bone by twisting handle 402 of inserter 400 or inserter/welding device 940 (see FIGS. 8A-SD). Alternatively, the inserter or inserter/welding device may be powered to drive the anchor base into the bone automatically.

After the anchor base 100 is inserted into the bone, the outer tube 404 may be slid backward relative to the inner tube 420 and handle 402. If the anchor top 200 has been positioned relative to the anchor base already, sliding the outer tube backward exposes the anchor top 200 as shown in FIG. 9B. Alternatively, the anchor top 200 may be moved into the position shown in FIG. 9B where it may be held in place as described above in connection with FIG. 6.

Once the anchor top 200 is positioned relative to the anchor base 100, with the stem of the anchor top extending into the central bore of the anchor base, one or more lengths of suture 600 may then be placed in the space between the distal surface 602 of the proximal member 204 of anchor top 200 and the proximal surface 604 of the anchor base 100 by moving the suture laterally into the space as depicted in FIG. 9C. The suture 600 may be manually tensioned as desired. In some embodiments, tensioning of the suture 600 is aided by pulling the suture 600 against the distal member 202 of the anchor top 200.

In an embodiment utilizing a draw wire 422 that extends out of inner tube 420, pressing wire manipulator 408 causes the handle 402 of the inserter 400 and the inner tube 420 to be pulled down towards the anchor base 100 as depicted in FIG. 9D. In another embodiment, the proximal member 204 of the anchor top 200 may be formed with a swage on the outer edge to be removeably coupled to the inner tube and guided into position over the anchor base 100. Alternatively, in an embodiment in which the anchor top is subsequently moved into position over the anchor base 100, the tip 414 of inserter/welding device 940 may be used to exert axial pressure on the anchor top 200. As the anchor top 200 is pushed axially down, suture 600 will be clamped between the distal surface 602 of the proximal member 204 of anchor top 200 and the proximal surface 604 of the anchor base 100 (see also FIG. 9C). The clamping will force the suture to be compressed within the passageways 302 and 304 depicted in FIG. 6B and thus be secured to anchor 300.

Once the anchor top 200 has been pressed into the anchor base 100, FIGS. 9E and 9F provide two example of how the welding device 900 (described above with reference to FIGS. 7A and 7B) or the inserter/welding device 940 (described above with reference to FIG. 8D) may be used to further secure a suture 600 to the non-metallic anchor 300 by welding the welding features of the non-metallic anchor top 200 to the anchor base 100.

Referring to FIG. 9E, the inserter 400 has been used to screw the non-metallic anchor base 100 into the bone as described and shown in FIG. 9A, and the suture has been placed between the distal surface 602 of the non-metallic anchor top 200 and the proximal surface 604 of the non-metallic anchor base 100 (as shown in FIGS. 9B-9D).

The tip 914 of the welding device 900 is pressed firmly against the proximal surface in the approximate location immediately above the internal welding features 230. With the tip 914 pressed firmly against the anchor top 200, the operator may activate the ultrasonic welder 900, thereby sending ultrasonic pulses through the proximal surface of the anchor top 200. The internal welding features 230, upon receiving the ultrasonic energy will flow into the interstices of the suture 600 below and into the proximal surface 604 of the non-metallic anchor base 200. When the flowing plastic becomes solid again, it forms a secure bond among the various components. As discussed above with reference to FIGS. 6A and 6B, in some alternative embodiments, external welding features may be used in addition to or instead of the internal welding features.

FIG. 9F depicts an alternate embodiment in which the inserter/welder 940 (discussed above with reference to FIG. 8D) may be used on a non-metallic anchor top 200 to weld interior or exterior welding features on the anchor top 200. The ultrasonic tip 421 extends out of the outer tube 404 to touch the proximal surface of the proximal member 204. In this illustration, the inserter functionality of the inserter/welder device 940 may be used to first screw the non-metallic anchor base 100 into the bone and press the non-metallic anchor top 200 into the anchor base as illustrated in FIGS. 9A-9D. With the ultrasonic tip 421 pressed against the anchor top 200, ultrasonic energy is then transmitted from the tip 421 into the anchor top 200 to weld one or more of the welding features. As noted previously, the welding features may be located anywhere on the distal portion of the proximal member of the anchor top 200, and need only be configured to melt into the suture 600, the non-metallic anchor base 100, or both in order to strengthen the bond holding the suture in place.

In yet another embodiment, a modified inserter/welder 940 may configured to operate on welding features that are located in the tip of distal member 202 of the non-metallic anchor 200. In this embodiment, an inner tube 425 disposed within inner tube 420 of the inserter/welding device 940 may have a circumference sufficiently narrow to be extended down into the hole 208 in the center of proximal member 204 of non-metallic anchor top 200. The inner-inner tube may further include ultrasonically conductive material through which ultrasonic energy may be transferred to its tip. The ultrasonic energy may also be transferred from the device 940 through the stem of the anchor top 200 in order to fuse the features at the tip of the distal member.

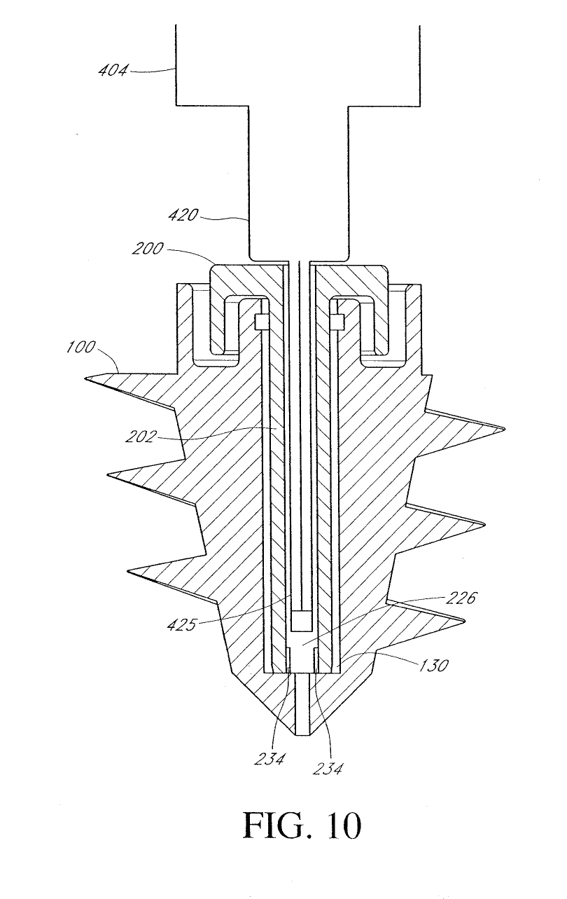

FIG. 10 depicts a cross section of a non-metallic anchor 300 including a non-metallic anchor top 200 with tip welding features 234 as shown above in FIG. 5D. The inner tube 420 of the inserter/welder device 940 is positioned above the proximal member 204. The inner-inner tube 425 extends into the hole 208 which leads to the central bore 130 of distal member 202.

At or near the bottom of the anchor top central bore 226, tip welding features 234 are positioned to receive the ultrasonic energy transmitted to the tip of the inner-inner tube 425 of the modified inserter/welder 940. When the welding features receive the ultrasonic energy, they flow from their solid state and bond with the exterior walls of the anchor base central bore 130.

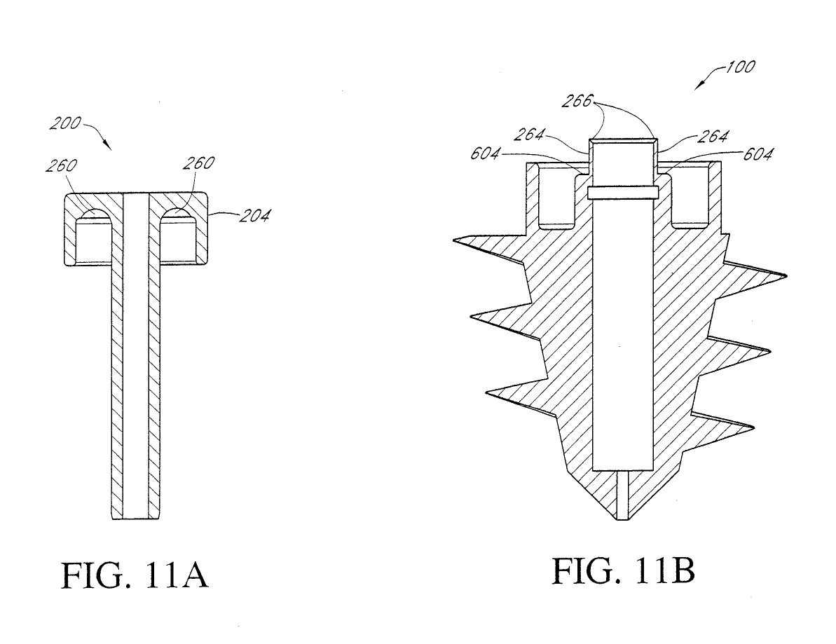

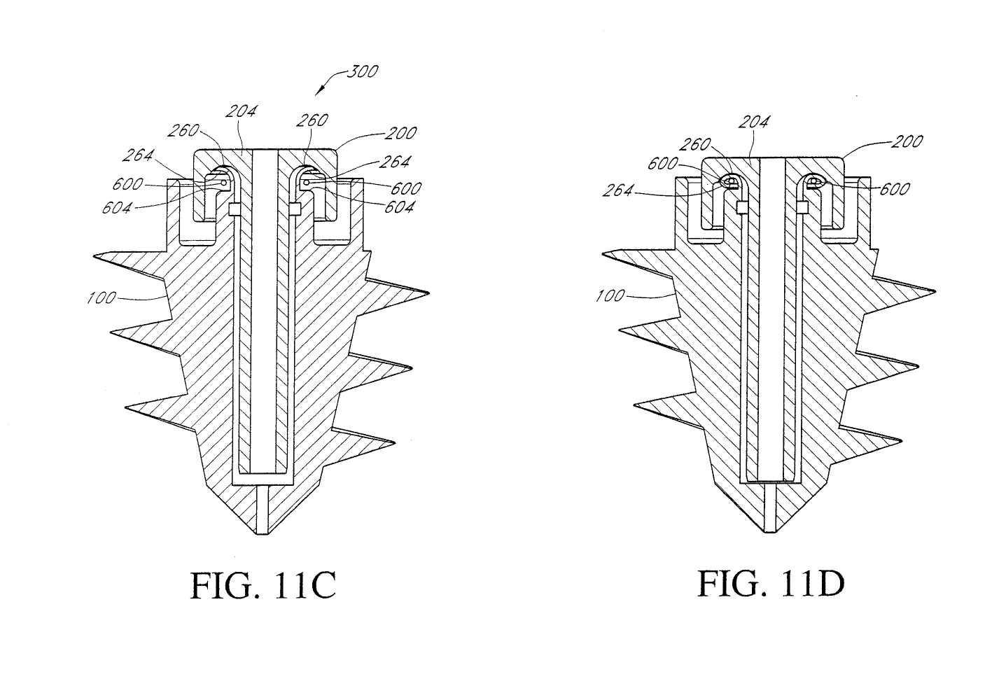

FIGS. 11A-11D depict yet another embodiment of a non-metallic anchor 300 made up of a non-metallic anchor base 100 and a non-metallic anchor top 200. The anchor depicted in FIGS. 11A-11D uses anvils and crimp sleeves as part of a swage-like securing mechanism for securing the suture 600 to the non-metallic anchor 300.

Referring now to FIG. 11A, a cross-section view of a modified non-metallic anchor top 200 is provided. The anchor top is generally similar to those described above, but further includes an anvil 260 on the distal surface of the anchor top proximal member 204. The anvil may be a rounded indentation in the distal surface, or it may take some other form that provides a crimping or shaping function as will be discussed in more detail below with reference to FIGS. 11B-11D.

FIG. 11B depicts a cross section view of non-metallic anchor base 100 with crimpable tubular extensions 264. The crimp sleeves may provide two functions. First, they may be used to further secure the suture. Second, they may also be used to further secure the anchor base 100 to the anchor top 200. The crimpable tubular extensions 264 may extend from the proximal surface 604 of the anchor base, and may be formed of a pliant material that can be rolled or deformed into a cylindrical shape. The proximal tips 266 of the crimpable tubular extensions 264 may be angled inward (as shown in FIG. l B) so that when they receive downward pressure from the anvil 260, they curl outward, away from the axial center of the anchor base 100. Alternatively, the crimpable tubular extensions 264 may be angled outward so that they curl inward toward the axial center of the anchor base 100.

Referring now to FIG. 11C, a cross section view of the non-metallic anchor base 100 receiving non-metallic anchor top 200 is provided. A suture 600 is positioned in the area between the distal portion of the anchor top proximal member 204 and the proximal surface 604 of the anchor base 100. The non-metallic anchor top 200 shown in FIG. 11C is shown in a partially inserted state. The anvil 260, which forms part of the underside of the anchor top proximal member 204, exerts downward pressure on the crimp sleeves 264 of the anchor base 100. Upon receiving the downward pressure from the anvil 260, the crimp sleeves 264 begin to deform by folding over, using the shape of the anvil as a mold or guide. As the crimp sleeves 264 deform, they form a coil around the suture 600.

FIG. 11D depicts a cross-section of the non-metallic anchor 300 with the anchor top 200 fully inserted into the anchor base 100 resulting in the full deformation of the crimp sleeves 264 around the suture 600. As the anchor top 200 is fully inserted into the anchor base 100, the crimp sleeves 264 continue to bend, and become a tightly wound cylinder around the suture 600 helping to secure it more firmly in place. In one embodiment, the proximal surface of the anchor base 100 may also include an anvil shaped indentation (not shown) directly underneath the anvil 260 on the distal surface of the anchor top 200, to provide a further shaping mechanism for the crimp sleeve 264 as it is pressed downward into the anchor base anvil. By forming the cylindrical shape around the suture, the suture may be advantageously protected from sharp edges in anchor 300 that could cause it damage. Once the crimp sleeve 264 has been fully deformed around the suture 600, modified inserter/welder 940 or ultrasonic welding device 900 may be used to weld the suture into the sleeve deformation, providing additional gripping strength for securing the suture 600.

In one alternative embodiment, only crimp sleeves 264 are non-metallic. The rest of the anchor 300 may be metallic. In still another embodiment, the crimp sleeve is located on a distal surface of the anchor top and the anvil is located on a proximal surface of the anchor base. In another embodiment, ultrasonic energy may be applied as described above to melt the crimp sleeves 264 to provide enhanced bonding with the suture 600. In some embodiments, the anchor top 200 is secured to the anchor base 100 using ultrasonic welding. In other embodiments, the anchor top 200 is secured to the anchor base 100 using a ratchet mechanism as described in U.S. Pat. application Ser. No. 11/143,007, which is hereby incorporated by reference.

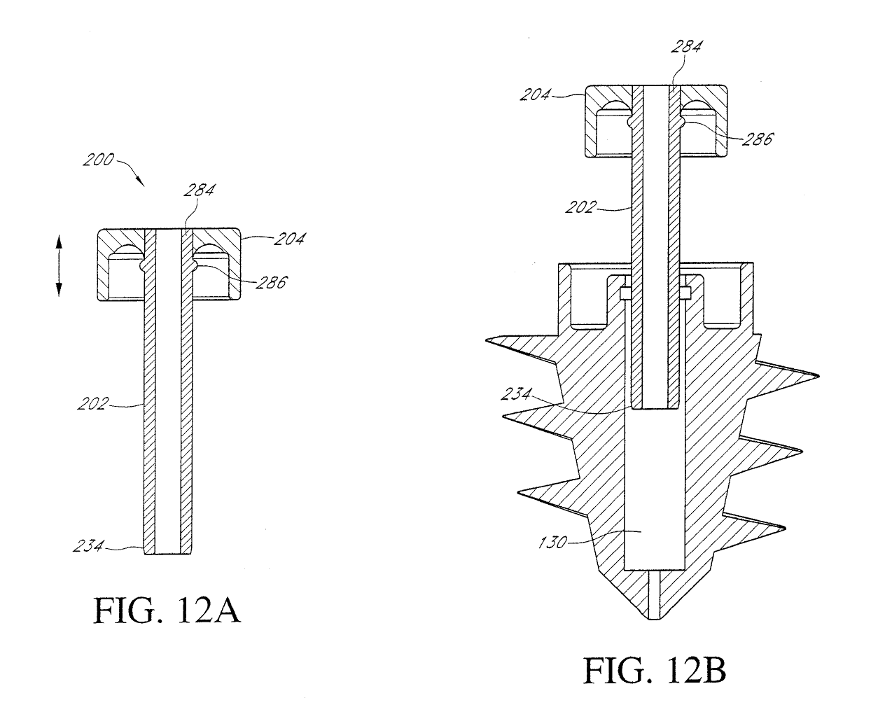

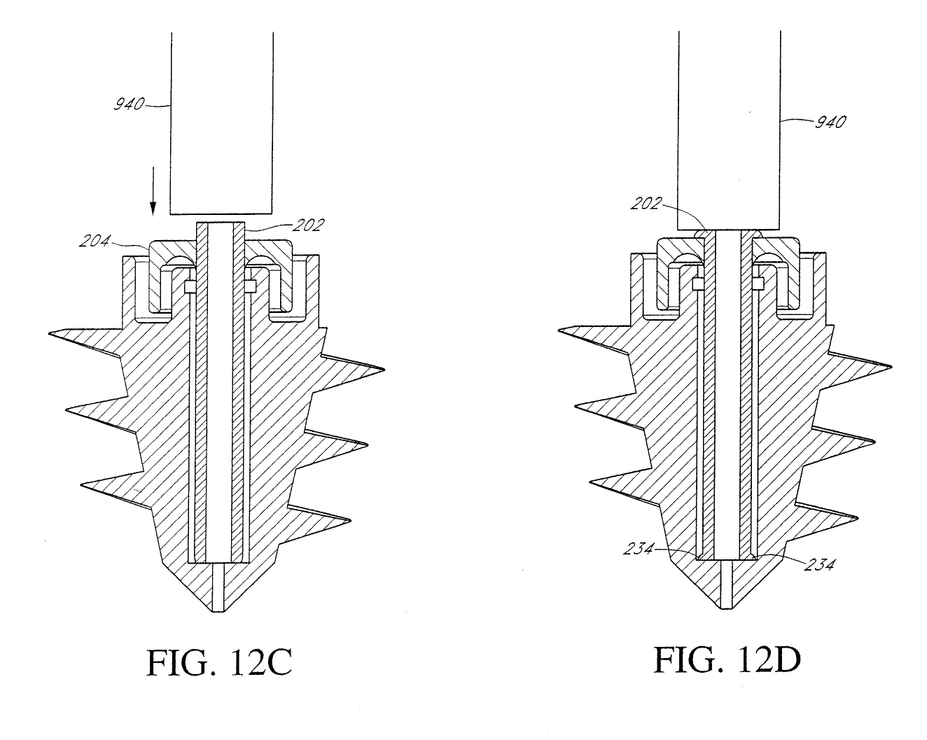

FIGS. 12A-12D depict yet another embodiment of a non-metallic anchor 300 made up of a non-metallic anchor base 100 and a non-metallic anchor top 200. The anchor depicted in FIGS. 12A-12D includes a “floating” anchor cap (or proximal member 204) of the anchor top 200 which may be slidably positioned on the distal member 202 to move up and down the shaft. The distal member 202 may include a pair of protrusions 286. The protrusions 286 prevent the slidable proximal member 204 from falling down the shaft-like distal member 202 which would make it difficult to slide a suture into a position for capture.

In one embodiment, the distal member 202 is configured to be a push fit with respect to the central bore 130 of the anchor base 100 as shown in FIG. 12B. In such a configuration, the distal member provides a sufficiently tight push fit that it stays stationary within the central bore 130 unless downward pressure is applied from above (e.g., on the proximal area).

Referring now to FIG. 12C, the anchor top 200 is shown as it is pushed down into the central bore 130 of the anchor base 100. In the embodiment shown, the anchor top 200 is pushed downward by the inserter/welding device 940. Other devices, however, may be used to apply the downward pressure. As the distal member 202 is pushed further into the central bore 130, the protrusions 286 may deform or be sheared off, which allows the anchor cap/proximal member 204 of the anchor top 200 to fall down and trap a suture that has been placed in the area below. Once the anchor cap 204 has slid down the shaft of the distal member 202, the top portion of the distal member 202 may be exposed.

Once the cap has slid down the shaft, the anchor top 200 may be secured to the anchor base 100 to trap a suture in the space between the proximal surface of the anchor base 100 and the distal surface of the proximal member 204 of the anchor top 200. Once the distal member 202 of the anchor top 200 is fully inserted into the central bore 130 of anchor base 100, the inserter/welding device 940 may be activated against to the exposed proximal portion of the distal member 202 causing the member to flow in both the proximal and distal areas. The portion of the distal member 202 adjacent to the inserter/welding device 940 is welded over the floating anchor cap 204, securing the anchor cap 204 in place relative to the shaft of the distal member 202. The tip welding features 234 located on the distal portion of the distal member shaft 202 also flow and weld to the exterior walls of the central bore 130 of the anchor base 100.

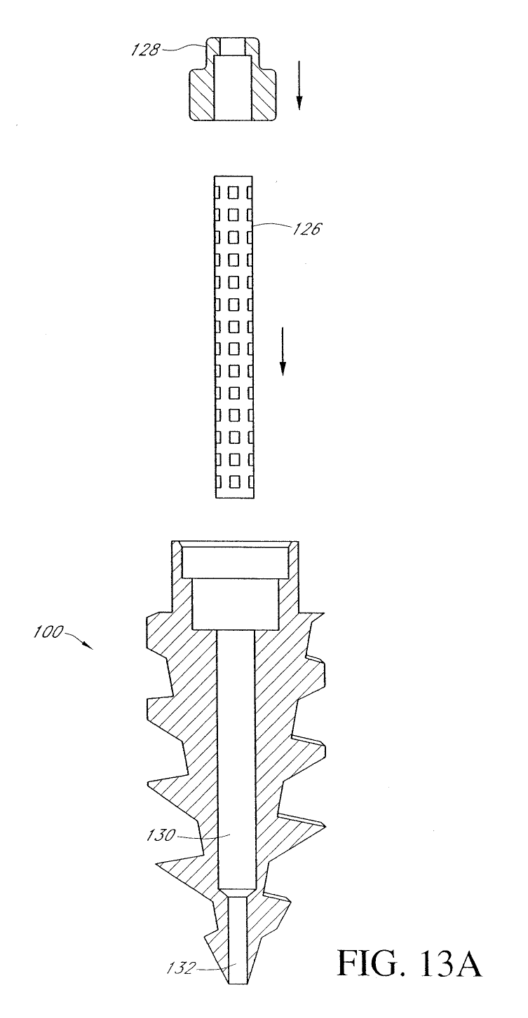

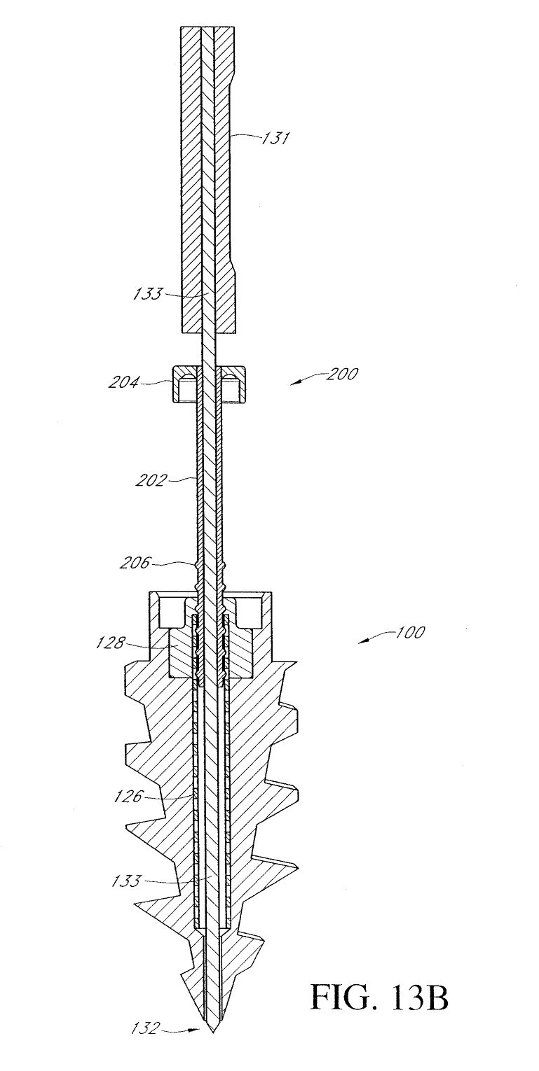

As noted previously, in some embodiments, portions of the anchor 300 may be non-metallic while other portions are metallic. Although these “hybrid” configurations do not eliminate all metal from the implant, substantial reductions in metal may be achieved. Referring now to FIG. 13A, a hybrid non-metallic anchor base 100 is provided. As discussed above, the anchor base 100 may include a central bore 130 and a smaller inner bore 132. A ratchet sleeve 126 is inserted into the central bore 130 by movement in the direction of arrow “1” and may be secured inside the anchor base 100 through the use of an insertable hub 128 which is moved into the anchor base 100 as shown by arrow “2”, and may be bonded or welded to the proximal surface of the anchor base 100 to capture the ratchet sleeve 126 inside the central bore 130. The ratchet sleeve 126 may be formed of various materials, including titanium or some other metallic substance or compound. Titanium provides advantages of reduced MRI artifacts as compared with other metals such as stainless steel.

FIG. 13B illustrates how the assembled anchor base 100 having a ratchet sleeve 126 in its central bore 130 (secured by insertable hub 128) may receive an anchor top 200 to secure suture material between the anchor top 200 and anchor base 100 as described above. The anchor top 200 may have a proximal member 204 and a distal member 202. A series of grooves or other mating or locking surfaces or structures 206 exist along a portion of the outside surface of the shaft of the distal member 202. The distal member 202 is designed to be inserted into the central bore 130 of the anchor base 100. The ratchet structures 122 in the anchor base 100 engage grooves 206 to couple the anchor top 200 to the anchor base 100. The ratchet structures 122 are oriented such that the distal member 202 can be easily moved in the distal direction in central bore 130 with the ratchet structures 122 snapping into the grooves 206 as the distal member 202 is moved downward. However, when the ratchet structures 122 are snapped into grooves 206, proximal movement of distal member 202 is inhibited. Thus, the anchor top 200 may be ratcheted down into anchor base 100. Because the ratchet structures 122 may exist along substantially the entire surface of the central bore 130 (see FIG. 13A) the anchor top 200 may be coupled to the anchor base 100 in several positions. In other words, in one embodiment the anchor top 200 need not be ratcheted into the anchor base 100 to its fullest extent in order for it to be secured to the anchor base 100.

In some embodiments, a draw wire 133 may be used to lock the anchor top 200 to the anchor base 100 using an inserting device. The smaller bore 132, which forms the tip of the anchor base 100, may be used to receive the wire. In some embodiments, the tip may be swaged to form an interference fit, and the draw wire may be welded inside the smaller bore 132. It will be appreciated that other structures than the smaller bore 132 may be used for attaching the wire 133 to the anchor base 100. In some embodiments, the anchor top 200, the ratchet sleeve 126, and the hub 128 may then be slid over the draw wire 133 at which point the wire may be connected to an anchor inserter as described above in connection with FIG. 8C. Alternatively, the draw wire may be secured to a draw bar 131. In some embodiments, the draw bar 131 may be positioned inside the inner tube 420 of the inserter/welder device 940 described above in relation to FIG. 8C and FIGS. 9C and 9D. The draw wire may be secured to the draw bar 131 by a welding process or by some other securing mechanism during manufacture of the anchor inserter assembly. The draw bar 131 may form a portion of an anchor inserter 400. The anchor inserter 400 may include a draw bar manipulator that may retract the draw bar 131 thereby deploying the anchor top 200 into the anchor base 100. When the draw bar 131 is retracted away from the anchor base 100, by the draw bar manipulator, the increase of tension on the wire may cause the wire may break free from the anchor 300 at its attachment point in bore 132 or at some other predetermined location along the wire.

Although the invention has been described with reference to embodiments and examples, it should be understood that numerous and various modifications can be made without departing from the spirit of the invention. Accordingly, the invention is limited only by the following claims.

What is claimed is:

1. An anchor for securing a suture to bone, comprising:
an anchor top having a welding feature; and
an anchor base configured to receive the anchor top.

2. The anchor top of claim 1, wherein at least a portion of the anchor top is non-metallic.

3. The anchor top of claim I, wherein at least a portion of the anchor base is non-metallic.

4. The anchor top of claim 1, wherein the anchor top comprises polyether ether ketone (PEEK).

5. The anchor top of claim 1 wherein the anchor top comprises polyether ketone (PEK).

6. The anchor top of claim 1, wherein the anchor base comprises polyether ether ketone.

7. The anchor of claim 1, wherein the anchor base is adapted to be securely fixed into the bone.

8. The anchor of claim 1, wherein the anchor top comprises at least part of a suture securing mechanism positioned proximally relative to the anchor base when the anchor top is coupled to the anchor base, the mechanism adapted to receive and secure a suture moved laterally into the mechanism.

9. The anchor of claim 8, wherein the suture securing mechanism comprises at least two surfaces adapted to secure a suture by welding of the two surfaces together.

10. The anchor of claim 9, wherein one of the surfaces is a proximal surface of the anchor base.

11. The anchor of claim 9, wherein a welding feature is located on one of the surfaces.

12. The anchor of claim 9, wherein the surfaces comprise the same non-metallic substance.

13. The anchor of claim 9, wherein one of the surfaces comprise a first non-metallic substance and the other of the surfaces comprise a different second non-metallic substance.

14. The anchor of claim 13, wherein the first non-metallic substance bonds with the second non-metallic substance when exposed to ultrasonic energy.

15. The anchor of claim 1, wherein the welding feature comprises a non-metallic protrusion from a distal facing surface of the anchor top.

16. The anchor of claim 15, wherein the welding feature comprises a material suitable for ultrasonic welding.

17. The anchor of claim 1, wherein the anchor base comprises lateral protrusions adapted to resist removal of the anchor base from bone.

18. The anchor of claim 17, wherein the lateral protrusions include threads adapted to allow the anchor base to be screwed into bone.

19. The anchor of claim 1, wherein the welding feature is located on a distal facing surface of the anchor top.

20. The anchor of claim 1, wherein the welding feature is located on a lateral facing surface of the anchor top.

21. The anchor of claim 1, wherein the anchor top comprises a proximal member and a distal member.

22. The anchor of claim 21, wherein the distal member comprises a shaft.

23. The anchor of claim 22, wherein the welding feature is located in the shaft.

24. A method of attaching soft tissue to bone, comprising:
passing a length of suture over the soft tissue;
inserting an anchor into the bone; and
securing the length of suture to the anchor after said inserting by ultrasonically welding an area of the anchor proximate to the suture.

25. The method of claim 24, wherein welding the area of the anchor proximate to the suture causes at least some of the anchor to flow into interstices of the suture.

26. The method of claim 24, wherein the securing occurs without passing an end of the length of suture through any aperture in the anchor and without tying any knots.

27. The method of claim 24, further comprising moving the length of suture laterally into a suture securing mechanism before securing it to the anchor.

28. The method of claim 24, wherein ultrasonically welding the area of the anchor proximate to the suture comprises:
applying pressure to the area of the anchor with an ultrasonic welding device; and
activating the ultrasonic welding device to send ultrasonic energy to the area of the anchor.

29. The method of claim 28, wherein the ultrasonic welding device is configured to both insert the anchor and ultrasonically capture the suture to the anchor.

30. The method of claim 28, wherein applying pressure to an area of the anchor comprises pressing a tip of the ultrasonic welding device against a proximal surface of the anchor.

31. The method of claim 30, wherein the anchor comprises an anchor top and an anchor base, and said proximal surface of the anchor is a proximal surface of the anchor top.

32. The method of claim 31, wherein the anchor top comprises a welding feature and the area of the anchor receiving the applied pressure is proximate to the welding feature.

33. The method of claim 31, further comprising:
securing the anchor top to the anchor base by inserting the ultrasonic welding device into a shaft in the anchor top; and
ultrasonically welding a stem of the anchor top to a central bore in the anchor base.

34. A device for inserting an anchor and securing a suture to the anchor, the device comprising:
means for inserting an anchor into bone; and
means for capturing a suture with the anchor by ultrasonically welding an area of the anchor proximate to the suture.

35. The device of claim 34, wherein the means for inserting the anchor comprises an inner tube, an outer tube, and a handle.

36. The device of claim 34, wherein the means for ultrasonically welding the suture comprises an ultrasonic welder.

37. An anchor for securing a suture to bone, comprising:
an anchor top having a crimp-shaping mechanism located on a distal surface of the anchor top; and
an anchor base having a crimpable extension adapted to deform upon receiving pressure from the crimp-shaping mechanism.

38. The anchor of claim 37, wherein the crimp-shaping mechanism comprises an anvil.

39. The anchor of claim 37, wherein the crimpable extension comprises PEEK.

40. The anchor of claim 37, wherein the anchor is configured to receive a suture in a location proximate to the crimpable extension, and wherein the crimpable extension is configured to deform around the suture upon receiving pressure from the anvil.

41. The anchor of claim 37, wherein the crimpable tubular extension comprises a material suitable for ultrasonic welding.

42. The anchor of claim 37, wherein the anchor base and the anchor top comprise a non-metallic substance.

43. The anchor of claim 37, wherein the crimp sleeve is a member protruding from the anchor base having a tip angled toward the axis of the anchor base, whereby when the crimp sleeve contacts the crimp-shaping mechanism, the crimp sleeve deforms in a direction away from the axis of the anchor base.

44. An anchor for securing a suture to bone, comprising:
an anchor top comprising:
a distal member having a protrusion on its exterior portion;
a proximal member slidably attached to the distal member; and
an anchor base having a central bore which receives the distal member.

45. The anchor of claim 44, wherein the protrusion is positioned to inhibit movement of the proximal member relative to the distal member.

46. The anchor of claim 45, wherein the protrusion is deformable.

47. The anchor of claim 45, wherein the protrusion is removable by exerting a force against the protrusion.

48. The anchor of claim 47, wherein inserting the distal member into the central bore causes a proximal surface of the anchor base to exert the force against the protrusion thereby causing the protrusion to be removed from the distal member.

49. The anchor of claim 47, wherein proximal member slides toward the proximal surface of the anchor base when the protrusion is removed from the distal member.

50. The anchor of claim 44, wherein the distal member comprises a material suitable for ultrasonic welding.

51. The anchor of claim 50, wherein the distal member bonds to the proximal member when exposed to ultrasonic energy.

52. The anchor of claim 51, wherein the distal member bonds to a central stem of the proximal member when exposed to ultrasonic energy.

53. The anchor of claim 44, wherein the distal member is a push fit relative to the central bore of the anchor base.

54. A device for inserting an anchor and securing a suture to the anchor, the device comprising:
an inserter portion configured to insert an anchor into bone; and
an ultrasonic welder portion configured to weld the suture to the anchor.

55. The device of claim 54, wherein the inserter portion comprises an inner tube, an outer tube, and a handle.

56. The device of claim 54, wherein the ultrasonic welder portion comprises an ultrasonic welder attached to the inner tube.
